(19) United States
(12) Patent Application Publication
(10) Pub. No.: US 2005/0215487 A1
(43) Pub. Date: Sep. 29, 2005

(54) Conjugates comprising an nsaid and a sugar and uses thereof

(76) Inventors: Michael F. Holick, Sudbury, MA (US); Halasya Ramanathan, Mysore (IL)

Correspondence Address: STERNE, KESSLER, GOLDSTEIN & FOX PLLC, 1100 NEW YORK AVENUE, N.W., WASHINGTON, DC, 20005, US

(21) Appl. No.: 10/518,618

(22) PCT Filed: Jun. 27, 2003
(86) PCT No.: PCT/US03/20188

§ 371 Date: Dec. 22, 2004

Related U.S. Application Data
(60) Provisional application No. 60/391,642, filed on Jun. 27, 2002.

(51) Int. Cl. A61K 31/70; A61K 31/7056

(52) U.S. Cl. 514/23; 536/18.7; 514/43

(57) ABSTRACT

Disclosed are conjugates comprising an NSAID and a sugar and uses thereof, e.g. for treating or preventing pain, fever and any condition characterized by an inflammatory process.

### BACKGROUND OF THE INVENTION

1. Field of the Invention

The present invention relates to conjugates comprising a nonsteroidal anti-inflammatory drug linked to a sugar and uses thereof.

2. Related Art

Nonsteroidal anti-inflammatory drugs (NSAIDs) are one of the most commonly prescribed groups of drugs worldwide and are highly effective as analgesic, antipyretic and anti-inflammatory agents. However, drugs of this class are responsible for substantial morbidity and mortality as a result of the complications associated with gastroduodenal ulcers, such as perforation and bleeding. The NSAIDs are nonselective cyclooxygenase inhibitors often experience dyspepsia and upper gastrointestinal (GI) adverse effects, and frequently require GI co-medications and diagnostic procedures.

It is estimated that 2% to 4% of patients who chronically use NSAIDs will present in a year's time with an UGI ulcer complication, such as bleeding, gastric outlet obstruction or perforation, and/or a symptomatic ulcer requiring the discontinuation of medications. It is further estimated that bleeding accounts for approximately 80% of NSAID-associated ulcer complications and that NSAID use results in about 107,000 hospitalizations and 16,500 deaths annually.

Previous clinical approaches for reducing the risks of ulcer complications or symptomatic ulcers in patients who use nonsteroidals have involved the coadministration of misoprostol or proton-pump inhibitors. Aspirin (as are NSAIDS) is an effective anti-inflammatory agent that inhibits cyclooxygenase. By a similar mechanism as found with NSAID use, aspirin is associated with significant UGI toxicity. Even using “so-called” low-dose aspirin (</=325 mg per day for cardiovascular prophylaxis), there is a 2- to 4-fold increase in the rate of UGI bleeding when compared with that occurring in age-matched patients not using aspirin. Unfortunately, there are little data demonstrating that this risk can be reduced by co-administration of protective medications. U.S. Pat. No. 4,965,065 describes gastroprotective processes and compositions comprising NSAIDs.

The UGI toxicity associated with aspirin and NSAID use is thought to be due to the inhibition of gastrointestinal mucosal cyclooxygenase (COX). COX catalyzes the conversion of arachidonic acid to prostaglandins, which mediate the development and maintenance of a physiologic protective barrier in the upper gastrointestinal tract. By inhibiting this key enzyme, mucosal protection is diminished and there is a resultant propensity for patients to develop gastroduodenal ulcers and ulcer complications.

Approximately 10 years ago, it was shown that COX exists as 2 isoforms: COX-1 and COX-2. COX-1 is constitutively expressed in the gastrointestinal tract and is responsible for maintaining mucosal protection; COX-2 is responsible for the inflammatory process. Because NSAIDs and aspirin inhibit both the isoforms somewhat equally at therapeutic doses, patients who use these medications are at risk for UGI ulcers and ulcer complications through the inhibition of COX-1. Over the past several years, a new class of agents known as the COX-2 inhibitors (celecoxib and rofecoxib) has been developed. These agents differ from the traditional NSAIDs in that they do not inhibit COX-1 at therapeutic doses. However, it is not clear whether patients taking COX-2 inhibitors have an actual smaller risk for UGI ulcers.

A number of systems have been developed to calculate the relative potency of the various NSAIDs against COX-1 and COX-2 including those using whole blood, recombinant enzymes and transfected cells. Results vary considerably depending on which system is used and there is little agreement on which should be the gold standard. Currently, a whole blood assay is the most widely accepted in vitro system. The definition of COX-2 selectivity also takes into account the in vivo and ex vivo activity of the NSAIDs.

Secondary amide derivatives of carboxylic acid —COOH containing NSAIDs, e.g. indomethacin, and their selective COX-2 action has been disclosed in U.S. Pat. No. 6,207,700. The amides were made starting mostly from secondary amines having aliphatic/aromatic groups. Particular preferred examples include indomethacin-N-methyl amide, indomethacin-N-ethan-2-ol amide, indomethacin-N-octyl amide, indomethacin-N-nonyl amide, indomethacin-N-(2-methylbenzyl) amide, indomethacin-N-(4-methylbenzyl) amide, indomethacin-N-((R)-,4-dimethylbenzyl) amide, indomethacin-N-((S)-,4-dimethylbenzyl) amide, indomethacin-N-(2-phenethyl) amide, indomethacin-N-(4-fluorophenyl) amide, indomethacin-N-(4-chlorophenyl) amide, indomethacin-N-(4-acetamidophenyl) amide, indomethacin-N-(4-methylmercapto)phenyl amide, indomethacin-N-(3-methylmercaptophenyl) amide, indomethacin-N-(4-methoxyphenyl) amide, indomethacin-N-(3-ethoxyphenyl) amide, indomethacin-N-(3,4,5-trimethoxyphenyl) amide, indomethacin-N-(3-pyridyl) amide, indomethacin-N-5-((2-chloro)pyridyl) amide, indomethacin-N-5-((1-ethyl)pyrazolo) amide, indomethacin-N-(3-chloropropyl) amide, indomethacin-N-methoxycarbonylmethyl amide, indomethacin-N-2-(2-L-methoxycarbonylethyl) amide, indomethacin-N-2-(2-D-methoxycarbonylethyl) amide, indomethacin-N-(4-methoxycarbonylbenzyl) amide, indomethacin-N-(4-methoxycarbonylmethylphenyl) amide, indomethacin-N-(2-pyrazinyl) amide, indomethacin-N-2-(4-methylthiazolyl) amide, and indomethacin-N-(4-biphenyl) amide.

U.S. Pat. Nos. 3,285,908 and 3,336,194 describe indoyl acid amides that are useful for the treatment of pain and inflammation. Particular indoyl amides include amides comprising an amino sugar. However, neither of these patents say anything about whether the amides have selective COX-1 or COX-2 inhibitory activities.

According to the present invention, by linking an NSAID to a sugar residue, one obtains a conjugate that offers many advantages. First and foremost, the sugar conjugate is expected to exhibit reduced and/or fewer side effects compared to the underivatized NSAID. When administered orally, the conjugate will be more soluble than the underivatized NSAID and, thus, is expected to cause less stomach irritation. Moreover, the NSAID will be in the form of a prodrug that will inhibit the COX-1 enzyme to a lesser extent or not at all compared to the underivatized NSAID. The conjugate will then be actively taken up by the GI tract, thus leading to lessened time in the GI tract. The bond between the NSAID and sugar is then cleaved in the blood and/or target tissue to release the NSAID in a slow and controllable fashion, thus leading to better pain management.

### SUMMARY OF THE INVENTION

The invention relates to compounds consisting of an NSAID linked to a sugar and uses thereof.

The present invention relates in particular to compounds of the Formula (I):
A-R¹ (I)
wherein A is the residue of an NSAID and R¹ is a sugar residue, with the proviso that said compound is not an indolyl acid amide.

The invention also relates to pharmaceutical compositions comprising the compounds of the invention and a pharmaceutically acceptable carrier.

The invention also relates to a method of treating or preventing pain, an inflammatory disease or cancer, comprising administering to an animal in need thereof an effective amount of a compound of the invention. As the many of the compounds of the invention are selective COX-2 inhibitors, preferably, the method is one for treating or preventing pain, an inflammatory disease or cancer with reduced and/or fewer side effects exhibited by the non-selective COX inhibitors.

The invention also relates to a method of preparing a compound of Formula (I), which comprises condensing a protected sugar with an NSAID. The protecting groups may then be partially or completely removed.

### DETAILED DESCRIPTION OF THE INVENTION

NSAIDs

The most commonly known group are the salicylates of which aspirin is the prime example. Another group of NSAIDs that have utility in connection with the instant invention are the proprionic acid derivatives. Included in this group, for example, are ibuprofen and naproxen. A further group of NSAIDs employable herein are the fenamates and compounds closely related to them structurally. These may be illustrated by such compounds as mefenamic acid, meclofenamic acid and diclofenac. Also belonging to the class of NSAIDs are the indole derivatives (e.g. indomethacin); pyrrolealkanoic acid derivatives (e.g. tolmetin); oxicams (e.g. piroxicam); and etodolac. The most preferred NSAIDs that may be used in the practice of the invention include the COX-2 inhibitors, e.g. the substituted pyrazolyl benzenesulfonamides such as celecoxib described in U.S. Pat. Nos. 5,466,823, 5,563,165, 5,760,068 and 5,972,986 as well as the diaryl-5-oxygenated-2-(5H)furanones such as rofecoxib described in U.S. Pat. Nos. 5,474,995, 5,691,374, 6,063,811 and 6,239,173.

Two naproxan-glucosamine conjugates of the invention have the formulae:

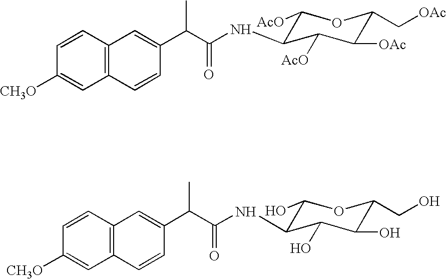

Other compounds of the invention have the formulae:

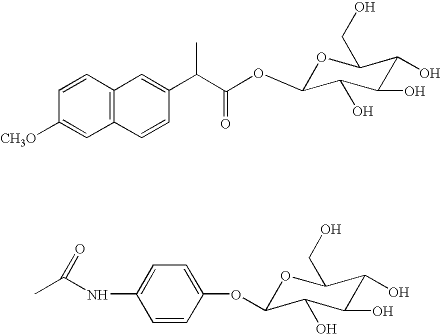

Where the NSAID is related to celecoxib, the compounds of the invention have the formula: 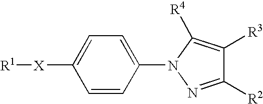
wherein R¹ is a sugar residue, X is selected from the group consisting of oxygen, —NH—, and —NS(O)₂—, R² is selected from hydrido, halo, haloalkyl, cyano, nitro, formyl, carboxyl, alkoxycarbonyl, carboxyalkyl, alkoxycarbonylalkyl, amidino, cyanoamidino, amido, alkoxy, amidoalkyl, N-monoalkylamido, N-monoarylamido, N,N-dialkylamido, N-alkyl-N-arylamido, alkylcarbonyl, alkylcarbonylalkyl, hydroxyalkyl, alkylthio, alkylsulfinyl, alkylsulfonyl, N-alkylsulfamyl, N-arylsulfamyl, arylsulfonyl, N,N-dialkylsulfamyl, N-alkyl-N-arylsulfamyl and heterocyclic; R³ is selected from hydrido, halo, haloalkyl, cyano, nitro, formyl, carboxyl, alkoxycarbonyl, carboxyalkyl, alkoxycarbonylalkyl, amidino, cyanoamidino, amido, alkoxy, amidoalkyl, N-monoalkylamido, N-monoarylamido, N,N-dialkylamido, N-alkyl-N-arylamido, alkylcarbonyl, alkylcarbonylalkyl, hydroxyalkyl, alkylthio, alkylsulfinyl, alkylsulfonyl, N-alkylsulfamyl, N-arylsulfamyl, arylsulfonyl, N,N-dialkylsulfamyl, N-alkyl-N-arylsulfamyl, heterocyclic, heterocycloalkyl and aralkyl; and R⁴ is selected from aryl, cycloalkyl, cycloalkenyl and heterocyclic; wherein R⁴ is optionally substituted at a substitutable position with one or more radicals selected from halo, alkylthio, alkylsulfinyl, alkyl, alkylsulfonyl, cyano, carboxyl, alkoxycarbonyl, amido, N-monoalkylamido, N-monoarylamido, N,N-dialkylamido, N-alkyl-N-arylamido, haloalkyl, hydroxyl, alkoxy hydroxyalkyl haloalkoxy, sulfamyl, N-alkylsulfamyl, amino, N-alkylamino, N,N-dialkylamino, heterocyclic, nitro and acylamino; or wherein R³ and R⁴ together form 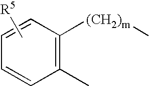
and m is 1 to 3, inclusive; and wherein R⁵ is one or more radicals selected from halo, alkylthio, alkylsulfinyl, alkylsulfonyl, cyano, carboxyl, alkoxycarbonyl, amido, N-monoalkylamido, N-monoarylamido, alkyl, N,N-dialkylamido, N-alkyl-N-arylamido, haloalkyl, hydrido, hydroxyl, alkoxy, hydroxyalkyl, haloalkoxy, sulfamyl, N-alkylsulfamyl, amino, alkylamino, heterocyclic, nitro and acylamino.

Preferably, R² and R³ are not identical radicals selected from hydrido, carboxyl and ethoxycarbonyl; further preferably R² cannot be carboxyl when R³ is hydrido and when R⁴ is phenyl; and further preferably R⁴ is sulfamyl or N-alkylsulfamyl when R¹ is halo; or a pharmaceutically-acceptable salt thereof.

Particular examples of compounds related to celecoxib that may be used to prepare the conjugates of the invention may found in U.S. Pat. No. 5,466,823. A particular example of a conjugate of the invention has the formula:

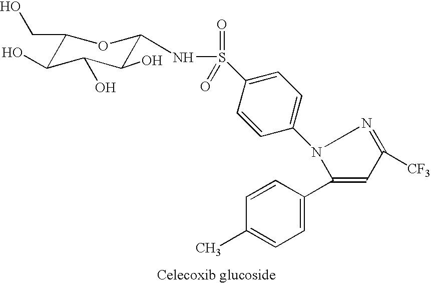

When the compounds are related to rofecoxib, they have the formula: 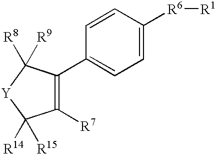
wherein Y is C(R₁₆)(R₁₇), oxygen, or sulfur; R⁶ is selected from the group consisting of oxygen, —NH—, and —NS(O)₂—; R⁷ is selected from the group consisting of (a) C₁₋₁₀ alkyl; (b) C₃₋₁₀ cycloalkyl; (c) C₂₋₁₀ alkenyl; (d) C₂₋₁₀ alkynyl; (e) C₃₋₁₀ cycloalkenyl;

(f) mono-, di-, tri- or tetra-substituted C₃₋₁₀ cycloalkenyl, wherein the substituent is selected from the group consisting of halo, C₁₋₆ alkoxy, C₁₋₆ alkylthio, CN, CF₃, C₁₋₁₀ alkyl, N₃, —CO₂H, —CO₂—C₁₋₁₀ alkyl, —C(R₁₀)(R₁₁)—OH, —C(R₁₀)(R₁₁)—O—C₁₋₄ alkyl, and C₁₋₁₀ alkyl-CO₂—R¹⁰; benzyloxy; —O—(C₁₋₁₀ alkyl)-CO₂R¹⁰; —O—(C₁₋₁₀ alkyl)-NR¹⁰R ¹¹;

(g) unsubstituted or mono-, di- or tri-substituted phenyl or naphthyl wherein the substituent is selected from the group consisting of C₁₋₁₀ alkyl, C₁₋₁₀ alkoxy, C₁₋₁₀ alkylthio, CN, CF₃, halo, N₃, —CO₂H, —CO₂-C₁₋₁₀ alkyl, C(R¹⁰)(R¹¹)—OH, —C(R¹⁰)(R¹¹)—O—C₁₋₄ alkyl, —C₁₋₆ alkyl-CO₂—R¹⁰, benzyloxy, —O—(C₁₋₁₀ alkyl)-CO₂R¹⁰, and-O—(C₁₋₁₀ alkyl)-NR¹⁰R¹¹;

(h) unsubstituted or mono-, di- or tri-substituted heteroaryl wherein the heteroaryl is a monocyclic aromatic ring of 5 atoms, said ring having one hetero atom which is S, O, or N, and optionally 1, 2, or 3 additional N atoms, or the heteroaryl is a monocyclic ring of 6 atoms, said ring having one hetero atom which is N, and optionally 1, 2, 3, or 4 additional N atoms, said substituents being selected from the group consisting of C₁₋₁₀ alkyl, C₁₋₁₀ alkoxy, C₁₋₁₀ alkylthio, CN, CF₃, halo, N₃, —CO₂H, —CO₂—C₁₋₁₀ alkyl, C(R¹⁰)(R¹¹)—OH, —C(R¹⁰)(R¹¹)—O—C₁₋₄ alkyl, —C₁₋₆ alkyl-CO₂—R¹⁰, benzyloxy, —O—(C₁₋₁₀ alkyl)-CO₂R¹⁰, and-O—(C₁₋₁₀ alkyl)-NR¹⁰R¹¹;

(i) an unsubstituted or a mono-, di-, tri- or tetra-substituted benzoheterocycle in which the heterocycle is a 5, 6, or 7-membered ring which may contain 1 or 2 heteroatoms chosen independently from O, S, or N and which may contain a carbonyl group or a sulfonyl group; the said substituents are selected from the group consisting of C₁₋₁₀ alkyl, C₁₋₁₀ alkoxy, C₁₋₁₀ alkylthio, CN, CF₃, halo, N₃, —CO₂H, —CO₂—C₁₋₁₀ alkyl, C(R¹⁰)(R¹¹)—OH, —C(R¹⁰)(R¹¹)—O—C₁₋₄ alkyl, —C₁₋₆ alkyl-CO₂—R¹⁰, benzyloxy, —O—(C₁₋₁₀ alkyl)-CO₂R¹⁰, and-O—(C₁₋₁₀alkyl)-NR¹⁰R¹¹;

(j) a heterocycloalkyl group of 5, 6 or 7 members which contains 1 or 2 heteroatoms chosen from O, S, or N and optionally contains a carbonyl group or a sulfonyl group;

(k) an unsubstituted or a mono- or di- substituted benzocarbocycle in which the carbocycle is a 5, 6, or 7-membered ring which optionally contains a carbonyl group, the said substituents are selected from the group consisting of C₁₋₁₀ alkyl, C₁₋₁₀ alkoxy, C₁₋₁₀ alkylthio, CN, CF₃, halo, N₃, —CO₂H, —CO₂—-C₁₋₁₀ alkyl, C(R¹⁰)(R¹¹)—OH, —C(R¹⁰)(R¹¹)—O—C₁₋₄ alkyl, —C₁₋₆ alkyl-CO₂—R¹⁰, benzyloxy, —O—(C₁₋₁₀ alkyl)-CO₂R¹⁰, and-O—(C₁₋₁₀ alkyl)-NR¹⁰R¹¹; R⁸ is hydrogen, C₁₋₁₀ alkyl, CH₂OR¹², CN, CH²CN, or C₁₋₆ fluoroalkyl, F, CONR¹²₂, unsubstituted or mono- or di-substituted phenyl, unsubstituted or mono or di-substituted benzyl, unsubstituted or mono- or di-substituted heteroaryl, unsubstituted or mono or di-substituted heteroarylmethyl, wherein the substituents are selected from the group consisting of C₁₋₁₀ alkyl, C₁₋₁₀ alkoxy, C₁₋₁₀ alkylthio, CN, CF₃, halo, N₃, —CO₂H, —CO₂—C₁₋₁₀ alkyl, C(R¹⁰)(R¹¹)—OH, —C(R¹⁰)(R¹¹)—O—C₁₋₄ alkyl, —C₁₋₆ alkyl-CO₂—R¹⁰, benzyloxy, —O—(C₁₋₁₀ alkyl)-CO₂R¹⁰, and-O—(C₁₋₁₀ alkyl)-NR¹⁰R¹¹; R⁹ is C₁₋₁₀ alkoxy, C₁₋₁₀ alkylthio, —OH, —OCOR¹², —SH, —SCOR¹², —OCO₂R¹³, —SCO₂R¹³, OCONR¹²₂, SCONR¹²₂, C₃₋₁₀ cycloalkoxy, C₃₋₁₀ cycloalkylthio; NR¹²₂; each R¹⁰ and R¹¹ is independently selected from the group consisting of hydrogen, and C₁₋₁₀ alkyl, or R¹⁰ and R¹¹ together with the carbon to which they are attached form a saturated monocyclic carbon ring of 3, 4, 5, 6 or 7 atoms; each R¹² is independently selected from the group consisting of hydrogen and R¹³; each R¹³ is independently selected from the group consisting of C₁₋₁₀ alkyl, phenyl or monosubstituted phenyl wherein the substituents may be halo, C₁₋₁₀ alkyl, C₁₋₁₀ alkoxy, C₁₋₁₀ alkylthio, CN, or CF₃; benzyl or monosubstituted benzyl wherein the substituents may be halo, C₁₋₁₀ alkyl, C₁₋₁₀ alkoxy, C₁₋₁₀ alkylthio, CN, or CF₃; and C₃₋₁₀ cycloalkyl; R¹⁴ and R¹⁵ are independently selected from the group consisting of: hydrogen, C₁₋₁₀ alkyl, and C₃₋₁₀ cycloalkyl, or R¹⁴ and R¹⁵ together form a double bonded O or S; R¹⁶ and R¹⁷ are independently selected from the group consisting of:

(a) hydrogen, (b) unsubstituted or mono- or di-substituted phenyl or unsubstituted or mono- or di-substituted benzyl or unsubstituted or mono- or di-substituted heteroaryl, or unsubstituted or mono- or di-substituted heteroarylmethyl, said substituents being selected from the group consisting of: C₁₋₁₀ alkyl, C₁₋₁₀ alkoxy, C₁₋₁₀ alkylthio, CN, CF₃, halo, N₃, —CO₂H, —CO₂—C₁₋₁₀ alkyl, C(R¹⁰)(R¹¹)—OH, —C(R¹⁰)(R¹¹)—O—C₁₋₄ alkyl, —C₁₋₆ alkyl-CO₂—R¹⁰, benzyloxy, —O—(C₁₋₁₀ alkyl)-CO₂R¹⁰, and-O—(C₁₋₁₀ alkyl)-NR¹⁰R¹¹; (c) C₁₋₁₀ alkyl, CH₂OR¹², CN, CH₂CN, C₁₋₁₀ fluoroalkyl, F or CONR¹²₂; or R¹⁶ and R¹⁷ together with the carbon to which they are attached form a carbonyl or a saturated monocyclic carbon ring of 3, 4, 5, 6 or 7 atoms; R¹⁸ and R¹⁹ are independently selected from the group consisting of: hydrogen and C₁₋₁₀ alkyl, or R¹⁸ and R¹⁹ together with the carbon to which they are attached form a carbonyl, —C(═S)—, or a saturated monocyclic carbon ring of 3, 4, 5, 6, or 7 atoms.

Particular examples of compounds useful to make the sugar conjugates of the invention may be found in U.S. Pat. No. 5,691,374.

Where the NSAID is related to ibuprofen, the compounds have the formula: 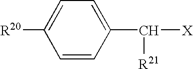
wherein R²⁰ represents ethyl, propyl, butyl (except n-butyl), pentyl (except n-pentyl), alkylene (C₂-C₄), alkoxy (C₂-C₃), phenoxy, phenylthio or cycloalkyl (C₅-C₇) optionally substituted by alkyl (C₁-C₂) in the 1-position, R²¹ represents hydrogen or methyl and X represents the radical COOR²¹ wherein R²² represents a sugar residue.

A preferred example is the glucosamide of ibuprofen:

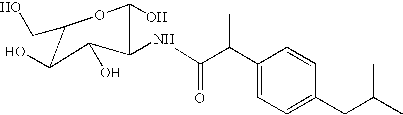

When the NSAID is related to mefenamic acid, meclofenamic acid and diclofenac, the compounds have the formula: 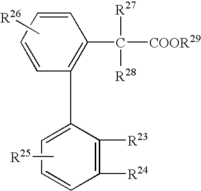
wherein:
R²⁵ is (lower) alkyl, (lower) alkoxy, fluoro or chloro;
each of R²³ and R²⁴ is hydrogen, (lower) alkyl, chloro or fluoro;
R²⁶ is hydrogen, (lower) alkyl, (lower) alkoxy, chloro, fluoro or bromo;
R²⁷ is hydrogen or (lower) alkyl;
R²⁸ is hydrogen, (lower) alkyl or when R⁵ is hydrogen, benzyl; and
R²⁹ is hydrogen, (lower) alkyl or benzyl. “Lower” means 1-4.

A particular example is the glucosamide of mefenamic acid having the formula:

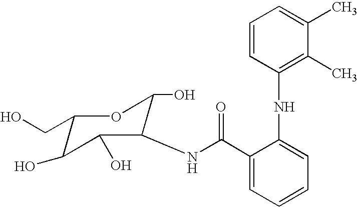

Another particular example is the glucosamide of meclofenamic acid having the formula:

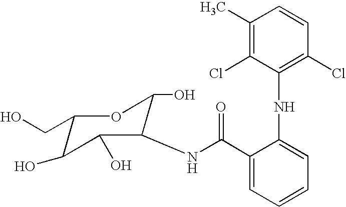

Another particular example is the glucosamide of diclofenac having the formula:

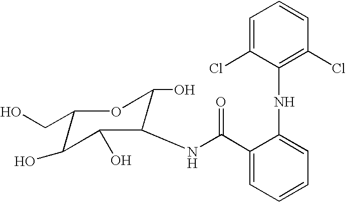

Where the NSAID is related to piroxicam, the compounds of the invention have the formula: 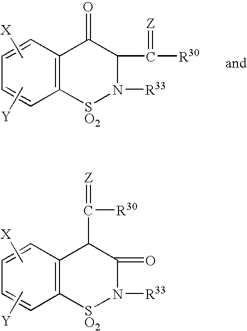
and the base salts thereof with pharmacologically acceptable cations, wherein X and Y are each a member selected from the group consisting of hydrogen, flourine, chlorine, bromine, nitro, alkyl and alkoxy having from one to five carbon atoms and trifluoromethyl; R³⁰ is a member selected from the group consisting of —OR³¹ and —NHR³², wherein R³¹ is alkyl having from one to twelve carbon atoms, phenylalkyl having up to three carbon atoms in the alkyl moiety or a sugar residue, and R³² is chosen from the group consisting of hydrogen, alkyl having from one to eight carbon atoms, alkenyl having up to six carbon atoms, cycloalkyl having up to eight carbon atoms, phenylalkyl having up to three carbon atoms in the alkyl moiety, nitrophenyl, naphthyl, phenyl, pyridyl, 3-methyl-2-pyridyl, 4-methyl-2-pyridyl, 5-methyl-2-pyridyl, 6-methyl-2-pyridyl, 4,6-dimethyl-2-pyridyl, 5-chloro-2-pyridyl, 5-bromo-2-pyridyl, 5-nitro-2-pyridyl, 3-hydroxy-2-pyridyl, 5-carboxamido-2-pyridyl, 2-pyrazinyl, 2-pyrimidyl, 4,5-dimethyl-2-pyrimidyl, 4-pyrimidyl, 5-methyl-3-pyridazinyl, 6-methoxy-3-pyridazinyl, 1-phenyl-3-pyrazolonyl, 2-triazolyl, 4-methyl-2-thiazolyl, 4,5-dimethyl-2-thiazolyl, 4-phenyl-2-thiazolyl, 5-bromo-2-thialazolyl, 3-isothiazolyl, 2-benzo-thiazolyl, 6-methyl-2-benzothiazolyl, 4-chloro-2-benzothiazolyl, 6-bromo-2-benzothiazolyl, 5-chloro-2-benzoxazolyl, 1,3,4-thiadazolyl, 5-methyl-1,3,4-thiadiazolyl, 1,2,4-thiazolyl, 6-phenyl-1,2,4-triazolyl, 7-indazolyl and mono- and di-substituted phenyl wherein each substituent is halogen, hydroxy, alkoxy and thioalkoxy having up to three carbon atoms, alkyl having up to four carbon atoms, trifluoromethyl, acetyl, methylsulfinyl or methylsulfonyl; R³³ is a member selected from the group consisting of hydrogen, alkyl having from one to six carbon atoms, alkenyl having up to four carbon atoms and phenylalkyl having up to three carbon atoms in the alkyl moiety, and Z is oxygen or sulfur, except when R³⁰ is OR³² when it is oxygen.

A particular example is the glucosamide of piroxicam:

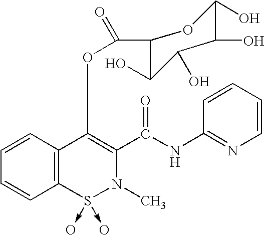

When the compounds of the invention are related to etodolac, they have the formula: 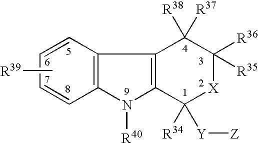
in which R³⁴ is selected from the group consisting of lower alkyl, lower alkenyl, lower alkynyl, lower cycloalkyl, phenyl, benzyl and 2-thienyl, R³⁵, R³⁶, R³⁷, and R³⁸ are the same or different and are each selected from the group consisting of hydrogen and lower alkyl, R³⁹ is selected from the group consisting of hydrogen, hydroxy, lower alkoxy, benzloxy, lower alkanoyloxy, nitro and halo, R⁴⁰ is selected from the group consisting of hydrogen, lower alkyl and lower alkenyl, X is selected from the group consisting of oxy and thio, Y is selected from the group consisting of carbonyl, 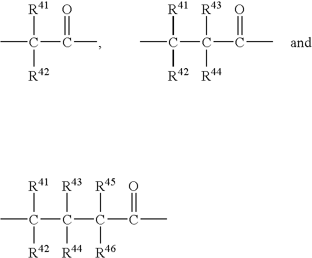
in which each of R⁴¹, R⁴², R⁴³, R⁴⁴, R⁴⁵ and R⁴⁶ is hydrogen or lower alkyl, and Z is selected from the group consisting of hydroxy, lower alkoxy, amino, lower alkylamino, di(lower)alkylamino and phenylamino.

A particular example is the glucosamide of etodolac:

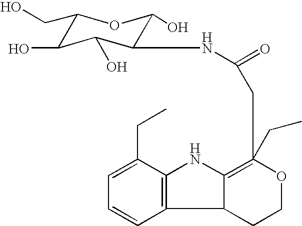

Other compounds of the invention include the sugar conjugates of aspirin such as the glucosamide having the formula: 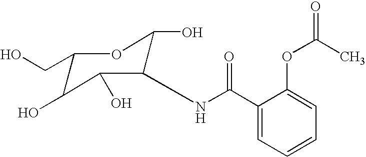
Sugar Residues

Sugar residues that are useful in the practice of the present invention include glucose, glucosamine, glucuronic acid, ribose, and the 2-deoxy derivatives thereof, e.g. 2-deoxy glucose, 2-deoxy-2-fluoroglucose and 2-deoxy ribose. In a preferred embodiment, the sugar residue is glucose which renders the conjugate more polar and water soluble, thus ameliorating any solubility problems of the NSAID.

Other sugar residues that may be used in the practice of the invention include derivatives of glucose, glucosamine and glucuronic acid. Preferably, endogenous glucosidases, glucuronidases, and amidases will recognize and cleave the sugar derivative-agent bond, thus releasing the NSAID. Also preferably, the sugar is 2-glucosamine and the compound of the invention is a glucosamide. It is expected that the amide compounds of the invention will be selective COX-2 inhibitors.

Particular examples of derivatives include the 2-fluoro derivatives, e.g. 2-fluoroglucose and 2-fluoroglucuronic acid.

The sugar residues may have free hydroxy groups, or the hydroxy groups may be acylated, e.g. with a group R₄—C═O)—, wherein R₄ is hydrogen, C₁₋₆ alkyl, C₆₋₁₀ substituted or unsubstituted aryl or C₇₋₁₆ aralkyl. Preferably, the acyl groups are acetyl or propionyl. Other preferred R⁴ groups are phenyl, nitrophenyl, halophenyl, lower alkyl substituted phenyl, lower alkoxy substituted phenyl and the like or benzyl, lower alkoxy substituted benzyl and the like.

The sugar residues may be fully or partially acylated or completely deacylated. The completely or partially acylated glycoside is useful as a defined intermediate for the synthesis of the deacylated material. Useful protecting groups include, but are not limited to, acetyl, benzoyl, nicotinoyl, benzyl, methyl and phenyl.

The compound of this invention can form an acid/base addition salt with an inorganic or organic acid or base.

Methods of Making the Compounds of the Invention

The NSAIDs may be conjugated to a protected glycose or glucuronide with an activated anomeric center such as halo, trichloro imidate, thiophenyl and their sulfoxides as is well known in the art. Glucuronic acid and glucosamine are preferred as they are stable molecules and are known to be cleaved by glucuronidase, glucosidase and amidases.

Amadori rearrangement is a main concern when linking sugars with amino containing compounds. This reaction is also called Malliard reaction.

However, the blocking of the anomeric position of the sugar avoids this adverse reaction. The anomeric position may be blocked with the NSAID having a hydroxy function (e.g. alcohols/phenols/carboxyl groups/enols) present on the agent. The coupling of the NSLAD with the hydroxy group of a sugar can be carried out with reagents such as EDC and DMT-MM {4-(4,6-dimethoxy-1,3,5-triazin-2-yl)-4-methylmorphol-inium chloride}. DMT-MM is more versatile than EDC, as the conjugation reaction may be carried out in protic solvents such as methanol, ethanol and water. Amide bond formation may be accomplished elegantly by this method.

Glucuronate/NSAID conjugates may be prepared by reacting protected glucuronic acid containing an activated anomeric position (e.g. the 1-halo, trimethylsilyl and tricholoroimidate derivatives) together with and the NSAID containing hydroxyl, phenolic and carboxyl functions. In another embodiment, an NSAID having an amino group may be conjugated to a glucuronic acid ester as shown in Scheme 1:

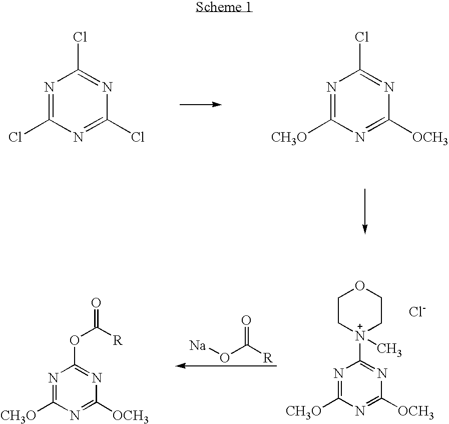

In another embodiment, 2-glucosamine-1,3,4,5-tetracetate is prepared then condensed with a carboxyl containing NSAID according to Scheme 2. 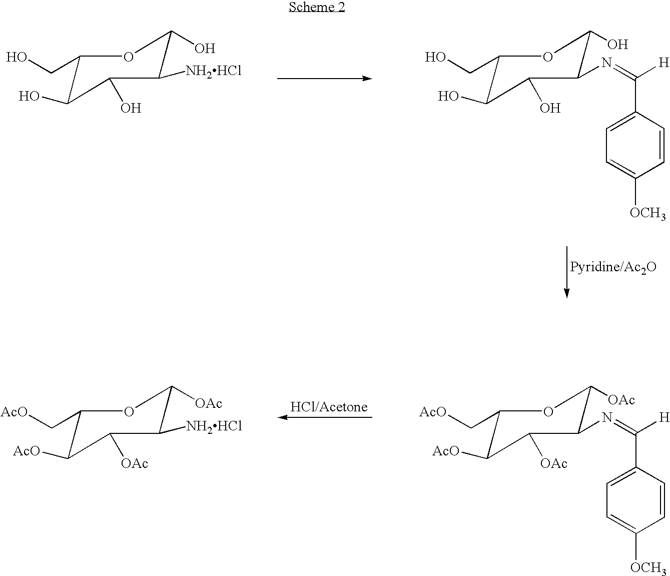
Methods of Use and Formulation

The compounds of the invention are useful for the treatment or prevention of pain, fever and any condition characterized by an inflammatory process including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, bums, injuries, following surgical and dental procedures. In addition, the compounds of the invention may inhibit cellular neoplastic transformations and metastic tumor growth and hence can be used in the treatment of cancer. Examples of such cancers include Hodgkin's disease, non-Hodgkin's lymphoma, acute lymphocytic leukemia, chronic lymphocytic leukemia, acute and chronic myelogenous lymphomas, multiple myeloma, neuroblastoma, breast carcinoma, ovarian carcinoma, lung carcinoma, Wilms' tumor, cervical carcinoma, testicular carcinoma, soft-tissue sarcoma, primary macroglobulinemia, bladder carcinoma, chronic granulocytic leukemia, primary brain carcinoma, malignant melanoma, small-cell lung carcinoma, stomach carcinoma, colon carcinoma, malignant pancreatic insulinoma, malignant carcinoid carcinoma, malignant melanoma, choriocarcinoma, mycosis fungoides, head or neck carcinoma, osteogenic sarcoma, pancreatic carcinoma, acute granulocytic leukemia, hairy cell leukemia, neuroblastoma, rhabdomyosarcoma, Kaposi's sarcoma, genitourinary carcinoma, thyroid carcinoma, esophageal carcinoma, malignant hypercalcemia, cervical hyperplasia, renal cell carcinoma, endometrial carcinoma, polycythemia vera, essential thrombocytosis, adrenal cortex carcinoma, skin cancer and prostatic carcinoma.

Compounds of the invention may also be useful for the treatment of dementia including pre-senile and senile dementia, and in particular, dementia associated with Alzheimer Disease (i.e. Alzheimer's dementia). In view of the improved side effects profile, the compounds of the invention are particularly useful for treatment of elderly patients exhibiting Alzheimer's disease, dementia or other conditions.

The compounds of the invention are expected to exhibit reduced side effects and/or incidence of side effects such as GI distress, GI ulcers, GI bleeding and the like. In a preferred embodiment, the compounds exhibit substantially no side effects, e.g. patients exhibit no GI distress, ulcers and bleeding when therapeutically effective doses are administered. Reductions in sides effects and incidence of side effects may be measured by administering the compounds of the invention and underivatized NSAIDs in equimolar amounts to statistically significant numbers of patients. A reduction of side effects will be noted when one or more of the well known side effects of NSAIDs are lessened, e.g. GI distress, ulcers and bleeding. A reduced incidence of side effects will be noted with one or more of the well known side effects of NSAIDs are not observed. However, it should be understood that when the invention is directed to a method, and wherein the method involves a reduction of side effects and/or incidence of side effects, it is intended that the method is practiced on individual patients as well as patient groups.

Particularly preferred routes of administration of the compounds of the present invention are per os, such as elixirs, tablets and capsules, as exemplified below, and by i.v. administration.

More generally, the compounds of the present invention can be administered in any appropriate pharmaceutically acceptable carrier for oral administration. The compounds of the invention may also be administered in any appropriate pharmaceutical carrier for parenteral, intramuscular, transdermal, intranasal, buccal or inhalation administration. They can be administered by any means that allow them to reach the target cells and tissues.

The dosage administered will depend on the age, health and weight of the recipient, the nature of the cancer, and the kind of concurrent treatment. An exemplary systemic dosage is about 0.1 mg to about 500 mg. Normally, from about 1.0 mg to 100 mg daily of the compounds, in one or more dosages before the diagnostic procedure, is effective to obtain the desired results. One of ordinary skill in the art can determine the optimal dosages and concentrations of active compounds with only routine experimentation.

The compounds can be employed in dosage forms such as tablets and capsules for oral administration. Such dosage forms may comprise well known pharmaceutically acceptable carriers and excipients. In a preferred embodiment, the dosage forms comprise cyclodextran and/or other saccharides and/or sugar alcohols. The compounds may also be formulated in a sterile liquid for formulations such as solutions (e.g. in saline) or suspensions for parenteral use. A lipid vehicle can be used in parenteral administration. The compounds could also be administered via topical patches, ointments, gels or other transdermal applications. In such compositions, the active ingredient will ordinarily be present in an amount of at least 0.001% by weight based on the total weight of the composition, and not more than 50% by weight. An inert pharmaceutically acceptable carrier is preferable such as 95% ethanol, vegetable oils, propylene glycols, saline buffers, sesame oil, etc. Remington's Pharmaceutical Sciences, 18^{th} Edition, Gennaro et al. (eds.), 1990, exemplifies methods of preparing pharmaceutical compositions.

The compounds may also be employed in fast dissolving dosage forms, as described in U.S. Pat. No. 6,316,027, comprising the compounds of the invention, water, gelatin and other ingredients.

The compounds of the invention may be formulated as part of a limposomal composition.

The compounds of the invention may be formulated together with a beta adrenergic agonist component, e.g. selected from the group consisting of isoproterenol, metaproterenol, terbutaline, albuterol, fenoterol, bitolterol, isoetharine, colterol, ritodrine, and their pharmaceutically acceptable salts, as described in U.S. Pat. No. 4,965,065.

Topical formulations for transdermal, intranasal or inhalation administration may be prepared according to methods well known in the art. For topical administration, the compounds may be applied in any of the conventional pharmaceutical forms. For example, the compounds may be administered as part of a cream, lotion, aerosol, ointment, powder, drops or transdermal patch. Ointments and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used include soft paraffin, aluminum stearate, cetostearyl alcohol, polyethylene glycols, wool-fat, hydrogenated lanolin, beeswax and the like.

Lotions may be formulated with an aqueous or oily base and will in general also include one or more of a stabilizing agent, thickening agent, dispersing agent, suspending agent, thickening agent, coloring agent, perfume and the like.

Powders may comprise any suitable powder base including talc, lactose, starch and the like. Drops may comprise an aqueous or non-aqueous base together with one or more dispersing agents, suspending agents, solubilizing agents and the like.

The compositions may further comprise one or more preservatives including bacteriostatic agents including methyl hydroxybenzoate, propyl hydroxybenzoate, chlorocresol, benzalkonium chloride and the like.

The topical compositions comprise from about 0.0001% to 5% by weight, preferably, 0.001 to 0.5% by weight, more preferably, 0.01 to 0.25% by weight of the active compounds.

The compounds of the invention are substantially pure. The phrase “substantially pure” encompasses compounds created by chemical synthesis and/or compounds substantially free of chemicals which may accompany the compounds in the natural state, as evidenced by thin layer chromatography (TLC) or high performance liquid chromatography (HPLC).

Animals which may be treated according to the methods of the present invention include all animals which may benefit therefrom. Included in such animals are humans, veterinary animals and pets, although the invention is not intended to be so limited.

### EXAMPLES

### Example 1

### Synthesis of 2-Chloro-4,6-dimethoxy-1,3,5-triazine

In a 2 liter jacketed reactor chilled to 0° C. equipped with an efficient mechanical stirrer were added methanol (12.3 moles; 393.5 g), water (2.7 moles; 49 g) and sodium bicarbonate (3 moles; 255 g). To this slurry was added cynauric chloride (183 g; 1 mole) at 10° C. in about 15 minutes. The stirred mixture was heated to 35° C. and maintained for 12 hours. Water (1.3 liters) was added at room temperature and the mixture was filtered off. The filtered cake was dried under vacuum. The dried material weighed (91 g; 60% yield). The melting point was in accordance with the literature (72-75° C.).

### Example 2

### Synthesis of 4-(4,6-dimethoxy-1,3,5-triazin-2-yl)-4-methylmorpholinium chloride (DMTMM)

N-Methylmorpholine (NMM; 111 g, 1.1 moles) was added over a period of 20 minutes to a stirred mixture of 2-Chloro-4,6-dimethoxy-1,3,5-triazine (175.5 g; 1 mole) dissolved in THF (1liter; commercial grade) at 10 to 15° C. The precipitate was filtered almost in quantitative yield, washed with THF (100 mL) and dried under vacuum. DMTMM was stored at −78° C. warmed to room temperature and used as and when needed.

### Example 3

### Synthesis of N-(p-methoxybenzylidene)-D-glucosamine

D-glucosamine hydrochloride (215 g; 1 mole) was dissolved in sodium hydroxide solution (1N; 1 liter ) and p-anisaldehyde (122 mL) was added. The solid product obtained was filtered off and dried. The product (250 g) had a melting point of 165° C. in accordance with the literature.

### Example 4

### Synthesis of N-(p-methoxy-benzylidene)-1,3,4,6-tetra-O-acetylD-glucosamine

The p-anisylidene derivative obtained above (250 g) was dissolved in pyridine (1.25 mL) and acetic anhydride (750 mL) was added slowly at room temperature. The mixture was stirred for 12 hours at room temperature and the clear solution was poured into crushed ice/water mixture (5 liters) and filtered. The precipitate was filtered off and crystallized from methanol (270 g). The product had a melting point of 180-1° C. in accordance with the literature.

### Example 5

### Synthesis of 1,3,4,6-tetra-O-acetyl-D-glucosamine hydrochloride

To a boiling solution of tetra-O-acetyl-p-anisylidene derivative (150 g) obtained as above in acetone (750 mL) was added hydrochloric acid (5N; 62.5 mL). After stirring the mixture mechanically for 15 minutes, the product was isolated by cooling and adding ether (100 mL) to facilitate complete precipitation. The precipitate was filtered and washed once with ether and dried (100 g; m.pt=230° C.)

### Example 6

### Synthesis of 2-(6-methoxy-2-napthyl)-propionyl-2′-D-glucosamide

D-glucosamine.hydrochloride (860 mg; 4 mMol) and naproxan (920 mg; 4 mMol) were suspended in methanol (20 mL) and sodium bicarbonate (670 mg) dissolved in 10 mL of water was added. The mixture was stirred for 15 minutes and DMTMM (1.1 g; 4 mMol) was added to the above mixture. The contents were stirred for a period of 15 hours at room temperature. The solvents were stripped under vacuum and the residue was chromatographed on silica gel using methanol-dichloromethane mixtures. The polar fractions containing the glucosamide were collected and evaporated under reduced pressure. The residue crystallized from isopropanol to afford the desired naproxan-glucosamide (875 mg).

NMR spectrum in deuterated methanol: δ (7.2-7.7; multiplet; 6-H; Aromatic-H); δ (3.7; singlet; methoxy; 3-H); δ (3.8-5.2; glucosyl-H and benzylic-H; 8H) and δ (1.4; overlapping doublets; 3-H; methyl)

The mass spectrum shows molecular ion at 414 amu as sodium ion adduct in accordance with the structure.

### Example 7

### Synthesis of 2-(6-methoxy-2-napthyl)-propionyl-2′-D-(31′,3′,4′,6′-tetra-O-acetyl)-glucosamide

Naproxan (1 g; 4.34 mMol) was dissolved in chloroform (75 mL) and stirred. 1,3,4,6-tetra-O-acetyl-glucosamine hydrochloride (2.2 g) and imidazole (500 mg) were added to the above mixture. The contents were stirred till all the glucosamine went into solution. Dicyclohexyl carbodiimide (1 g) was added and the contents stirred at room temperature for 12 hours. Dicyclohexyl urea was filtered off and the chloroform solution was evaporated and chromatographed on silica gel using ethyl acetate-hexane mixtures. 2-(6-methoxy-2-napthyl)-propionyl-2′-D-(1′,3′,4′,6′-tetra-O-acetyl)-glucosamide was obtained as a white powder (1.25 g). The spectral characteristics of the compound was in accordance with the structure.

NMR spectrum in CDCl₃: δ (7.2-7.7; multiplet; Aromatic-H; 6-H); δ (5.6-4.0; multiplet; glucosyl-H; 7-H); δ (3.9; methoxy; 3-H); δ (3.5-3.7; 2-H; glucosyl-H and benzylic-H), δ (1.9-2.1; overlapping singlets; acetyl; 12-H) and δ (1.5; complex overlap; methyl; 3-H)

The mass spectra showed molecular ion at 583 amu as a sodium ion adduct in accordance with the theoretical value of 582.54amu.

The deacetylation of the protecting groups in the glucosamine moiety was as follows.

### Example 8

### Deacetylation of 2-(6-methoxy-2-napthyl)-propionyl- 2′-D-(1′,3′,4′,6′-tetra-O-acetyl)-glucosamide

2-(6-methoxy-2-napthyl)-propionyl-2′-D-(1′,3′,4′,6′-tetra-O-acetyl)-glucosamide (800 mg) dissolved in methanol (40 mL) containing Dowex-550-OH resin (10 g; wet) was refluxed for 5 hours and filtered hot. Methanol was stripped off under reduced pressure and the product crystallized from isopropanol as before which afforded the desired glucosamide as a white powder (480 mg). The spectral characteristics were identical to the material obtained as in an earlier example.

From the foregoing description, one skilled in the art can easily ascertain the essential characteristics of this invention, and without departing from the spirit and scope thereof, can make various changes and modifications of the invention to adapt it to various usages and conditions without undue experimentation. All patents, patent applications and publications cited herein are incorporated by reference in their entirety.

1. A compound consisting of a non-steroidal antiiflammatory drug (NSAID) linked to a sugar, with the proviso that said compound is not an indolyl acid amide.

2. The compound of claim 1, wherein said NSAID is naproxan.

3. The compound of claim 1, wherein said NSAID is acetaminophen.

4. The compound of claim 1, wherein said NSAID is a substituted pyrazolyl benzenesulfonamide.

5. The compound of claim 4, wherein said substituted pyrazolyl benzenesulfonamide is celcoxib.

6. The compound of claim 1, wherein said NSAID is a diaryl-5-oxygenated-2-(5H)-furanone.

7. The compound of claim 6, wherein said diaryl-5-oxygenated-2-(5H)-furanone is rofecoxib.

8. The compound of claim 1, wherein said sugar is glucose, glucosamine, glucuronic acid, ribose, or the 2-deoxy derivatives thereof.

9. A pharmaceutical composition comprising the compound of claim 1 and a pharmaceutically acceptable carrier.

10. A method of treating or preventing pain, fever, condition characterized by an inflammatory process or cancer, comprising administering to an animal in need thereof an effective amount of a compound consisting of a non-steroidal antiiflammatory drug linked to a sugar, with the proviso that said compound is not an indolyl acid amide.

11. A method of treating or preventing pain, fever, condition characterized by an inflammatory process or cancer, with reduced and/or fewer side effects, comprising administering to an animal in need thereof an effective amount of a compound consisting of an NSAID linked to a sugar, whereby the animal is treated or the condition is prevented with reduced and/or fewer side effects compared to when the corresponding underivatized NSAID is administered to the animal.
